(19) United States
(12) Patent Application Publication
(10) Pub. No.: US 2026/0137496 A1
(43) Pub. Date: May 21, 2026

(54) SPRAYER DEVICE ADAPTABLE TO A DENTAL THREE-WAY SYRINGE

(71) Applicant: LISANDRO JESÚS HERRERA, Bariloche (AR)

(72) Inventor: LISANDRO JESÚS HERRERA, Bariloche (AR)

(21) Appl. No.: 19/393,694

(22) Filed: Nov. 19, 2025

(30) Foreign Application Priority Data
Nov. 21, 2024 (AR) 20240103214

(51) Int. Cl. A61C 19/06; A61C 1/00; A61C 1/05; A61M 11/06

(52) U.S. Cl. CPC A61C 19/063; A61C 1/0061; A61C 1/052; A61M 11/06

(57) ABSTRACT

A sprayer device adaptable to a dental three-way syringe for the controlled and nebulized administration of various dental solutions by a system that utilizes the existing compressed air flow in the syringes. The device permits the precise and directed application of dental solutions in both intraoral and extraoral zones, while maintaining the original functionality of the three-way syringe regarding the administration of water and air. Additionally, the device includes a system of interchangeable containers which allows for the sequential use of different dental solutions without needing to interrupt the clinical procedure to change instrumentation.

### FIELD OF THE INVENTION

The present invention relates to the field of medical-dental devices, and more particularly refers to a sprayer device that couples to conventional dental three-way syringes to allow for the controlled administration of solutions for professional use. Specifically, the invention pertains to the field of accessories and dispensing systems for dental products that complement the existing instrumentation in dental equipment, providing new functionalities for the application of therapeutic, antiseptic, anesthetic, and other types of solutions used in daily dental practice.

### PRIOR ART AND PROBLEMS TO BE SOLVED

In modern dental practice, the three-way syringe constitutes a fundamental tool present in all dental units and care offices. This widely known and used device is characterized by its ability to deliver air, water, or a combination of both, through an ergonomic handpiece connected to the unit's pneumatic and hydraulic system.

The conventional three-way syringe comprises a generally metallic main body that houses two independent conduits: one intended for the passage of water and another for the passage of compressed air. These conduits converge at a tip or terminal nozzle that allows the exit of said fluids. The control of the air and water flow is achieved using two independent push-buttons or buttons located on the syringe body, which can be actuated individually or simultaneously depending on the needs of the procedure. These buttons are responsible for actuating the valves of the respective independent conduits.

The ergonomic design of this device allows for one-handed manipulation, so that the dentist can selectively control the flow of water, air, or both, while holding another tool in the other hand.

Modern dental units are equipped with pumps and compressor equipment to provide the pressurized fluids, which are directed to the three-way syringe through a flexible hose.

As for the three-way syringe nozzle, since it is the part that comes into contact with the patient's mouth, it is designed to be detachable from the main body so it can be sterilized.

Naturally, like most dental instruments and tools, the components of three-way syringes, particularly the tips, are fabricated from surgical-grade stainless steel or other alloys suitable for medical use.

This nozzle primarily comprises two concentric conduits: the central conduit allows the passage of water, while the outer conduit allows the passage of compressed air. This configuration allows for the nebulization of water when both valves are actuated, allowing flow through both conduits.

Current three-way syringes permit the coupling of nozzles by means of threaded or press-fit connections.

This device constitutes a highly versatile and useful tool for a wide variety of operations typical of dental practice. For example, its most habitual use comprises tasks such as cleaning and washing the dental surface to remove debris, or cleaning the operative field during cavity preparations; drying dental surfaces prior to adhesive procedures; and/or removing debris during tooth preparation. Moreover, it is also highly useful for performing examinations, such as conducting pulp vitality tests via thermal stimulus by applying cold air; visualization of subgingival margins by means of drying; or the identification of fissures or fractures through selective drying. In surgical procedures, it is also indispensable for, for example, the irrigation of post-extraction sockets; or cooling during osteotomies; among other functions.

In daily dental practice, there is a frequent need to apply various solutions for professional use, such as topical anesthetics, antiseptics, fluoridation agents, bleaching solutions, desensitizers, and other specific chemical products. However, the current configuration of three-way syringes limits their functionality exclusively to the application of air and water, requiring these other solutions to be administered by means of additional instruments or devices.

Traditionally, the administration of dental solutions is carried out through the use of manual applicators, with one of the most common methods being the employment of cotton pellets. This method involves soaking the cotton in the desired solution and transporting it by means of forceps to the application site. Although this technique is simple, it presents significant disadvantages in terms of precision and control, as the quantity of solution administered depends entirely on the saturation of the cotton and the pressure exerted by the practitioner during application.

Another widely used method involves the use of disposable micro-applicators, which consist of small brush-type applicators that allow for the transport of reduced quantities of solution. Although these devices offer greater precision than cotton pellets, they require multiple refills to cover extensive areas, which prolongs the operative time and may result in a non-uniform application of the product. Furthermore, both cotton pellets and micro-applicators present significant limitations in reaching areas of difficult access within the oral cavity.

In the particular case of topical anesthetic solutions and other specific products, it is common for manufacturers to provide their own applicator devices, generally in the form of impregnated swabs or special applicator tips. This multiplicity of application systems compels the practitioner to maintain and handle various devices during the clinical procedure, which not only increases the operative complexity but also increases the risk of cross-contamination during instrument changes.

For the application of solutions in larger quantities, such as antiseptics or irrigation solutions, the use of conventional disposable syringes is frequently employed. However, these syringes are designed for the administration of liquids in the form of a stream and do not permit a nebulized or finely controlled application of the product. This is particularly problematic when a uniform distribution of the solution over an extensive surface is required or when seeking to minimize the risk of chemical splashes.

An essential disadvantage of current methods resides in the impossibility of achieving a nebulized and controlled application of the solutions, similar to that which is attained with water in conventional three-way syringes. This limitation is particularly relevant when a uniform distribution of the product over dental surfaces or soft tissues is required, or when seeking to minimize excess product that could be irritating to the surrounding tissues.

The lack of a unified and efficient system for the administration of these solutions not only affects the precision and control of the procedure, but also negatively impacts the economy of materials. The inability to accurately dose the solutions frequently results in product waste, which increases operating costs and may have implications in terms of environmental sustainability, especially considering the use of disposable devices.

Additionally, current methods require the practitioner to frequently interrupt the procedure to reload or change applicator devices, which not only prolongs the operative time but also may compromise the operator's concentration and the continuity of the treatment. This situation becomes particularly problematic in procedures that require the sequential application of different solutions, where each product change implies a change of applicator device.

### OBJECT OF THE INVENTION

It is an object of the present invention to provide a sprayer device adaptable to a dental three-way syringe for the administration of solutions for professional use, which solves the problems of the prior art. More particularly, it is an object of the present invention to provide a device that couples to conventional dental three-way syringes, permitting the controlled and nebulized administration of various dental solutions by means of a system that utilizes the existing compressed air flow in said syringes.

It is another object of the present invention to provide a device that allows for the precise and directed application of dental solutions in both intraoral and extraoral zones, while maintaining the original functionality of the three-way syringe regarding the administration of water and air.

It is a further object of the present invention to provide a system of interchangeable containers that permits the sequential utilization of different dental solutions without the need to interrupt the clinical procedure to change instruments.

It is also an object of the present invention to provide a device that optimizes the consumption of dental solutions by means of a controlled dosing system, reducing product waste and improving efficiency in professional practice.

### BRIEF DESCRIPTION OF FIGURES

FIG. 1A is a perspective view of the main body of the sprayer device adaptable to a dental three-way syringe in accordance with an embodiment of the present invention.

FIG. 1B is a front view of the main body of the sprayer device adaptable to a dental three-way syringe in accordance with an embodiment of the present invention.

FIG. 1C is a side view of the main body of the sprayer device adaptable to a dental three-way syringe in accordance with an embodiment of the present invention

FIG. 2A is a perspective view of the nebulizing spray nozzle of the sprayer device adaptable to a dental three-way syringe in accordance with an embodiment of the present invention.

FIG. 2B is a cross-sectional view of the nebulizing spray nozzle of the sprayer device adaptable to a dental three-way syringe in accordance with an embodiment of the present invention.

FIG. 2C is a detail view of the tip of the cross-section of the nebulizing spray nozzle of the sprayer device adaptable to a dental three-way syringe in accordance with an embodiment of the present invention.

FIG. 3A is a perspective view of the sprayer device adaptable to a dental three-way syringe in accordance with an embodiment of the present invention, mounted on said dental three-way syringe.

FIG. 3B is a side view of the sprayer device adaptable to a dental three-way syringe in accordance with an embodiment of the present invention, mounted on said dental three-way syringe.

FIG. 4A is a perspective view of the sprayer device adaptable to a dental three-way syringe in accordance with an embodiment of the present invention, mounted on said dental three-way syringe with an interchangeable container of dental solutions.

FIG. 4B is a side view of the sprayer device adaptable to a dental three-way syringe in accordance with an embodiment of the present invention, mounted on said dental three-way syringe with an interchangeable container of dental solutions.

FIG. 4C is a rear cross-sectional view of the sprayer device adaptable to a dental three-way syringe in accordance with an embodiment of the present invention, mounted on said dental three-way syringe with an interchangeable container of dental solutions.

### DETAILED DESCRIPTION OF THE INVENTION

In accordance with the present invention, there is provided a sprayer device adaptable to a dental three-way syringe which fundamentally comprises a main body (100) that couples to the conventional three-way syringe (300), a nebulizing spray nozzle (200), and a system of interchangeable containers (400).

The main body (100) of the device is configured to couple firmly and hermetically to the end of a conventional three-way syringe (300).

This main body (100) is preferably fabricated from surgical-grade stainless steel or anodized aluminum, materials selected for their biocompatibility and corrosion resistance, which makes them suitable for medical practice. Internally, the main body houses a system of conduits which, in addition to the air and water pathways corresponding to those originating from the conventional three-way syringe (300), includes a third pathway. Thus, an extra conduit is incorporated through which a dental solution such as topical anesthetics, antiseptics, fluoridation agents, bleaching solutions, desensitizers, among others, can be dispensed.

Housed within the main body (100) is an arrangement of concentric channels, wherein the central conduit is intended for the passage of water; the intermediate conduit is intended for the passage of compressed air; and the outer conduit is intended for the passage of dental solutions. Particularly, the outer conduit for the passage of dental solutions is arranged in such a way that the intermediate conduit for the passage of compressed air produces a Venturi suction effect.

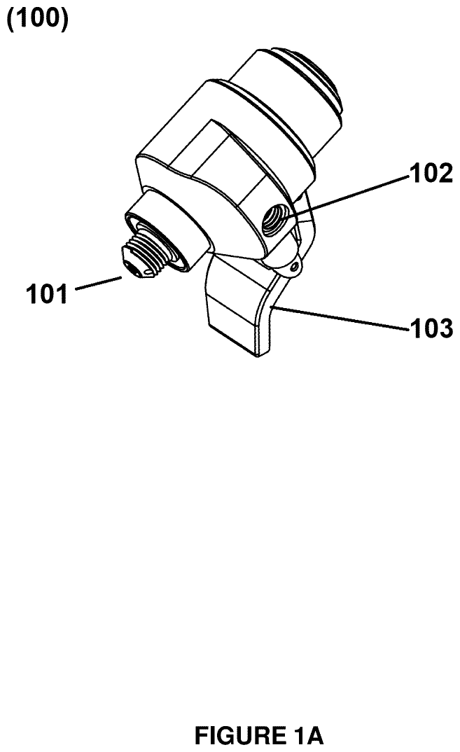

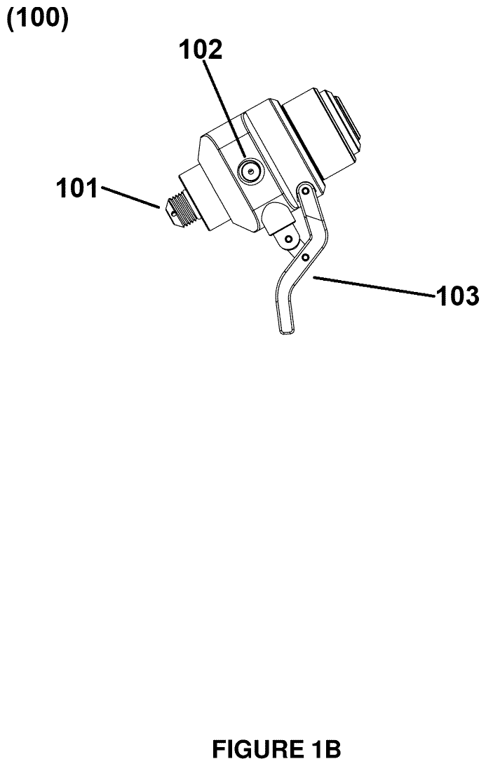

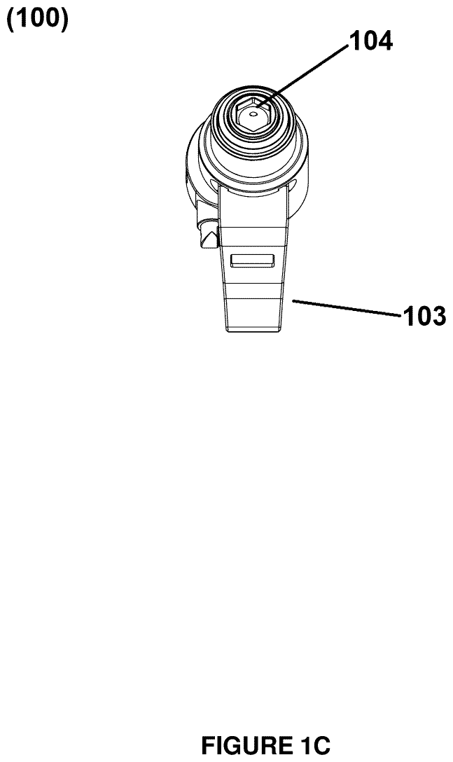

The connection system (101) of the main body (100) to the three-way syringe (300), as shown in FIGS. 1A-C, comprises a hermetic coupling mechanism that ensures the stability of the device during its use. The coupling (101) is effected in the same manner as a standard traditional nozzle of those commonly used in three-way syringes. Depending on the type and model of the three-way syringe to be used, the couplings can be press-fit or by means of a threaded union (101), as, for example, in the embodiment illustrated in FIGS. 1A-C. This system includes sealing elements, preferably O-rings made of elastomeric material, suitably disposed to prevent leaks and maintain tightness at the connection points. The coupling mechanism (101) is designed to maintain the precise alignment of the device's conduits with the original air and water pathways of the three-way syringe (300), thereby ensuring that the original functionality of the instrument is not compromised. In this manner, the attachable device does not limit the use of the three-way syringe (300), which can continue to be used normally in its original functions, but rather provides an additional function to be employed at the practitioner's discretion.

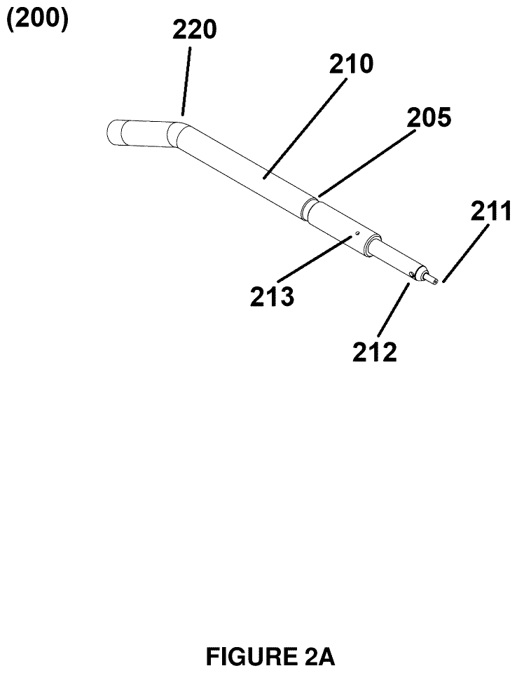

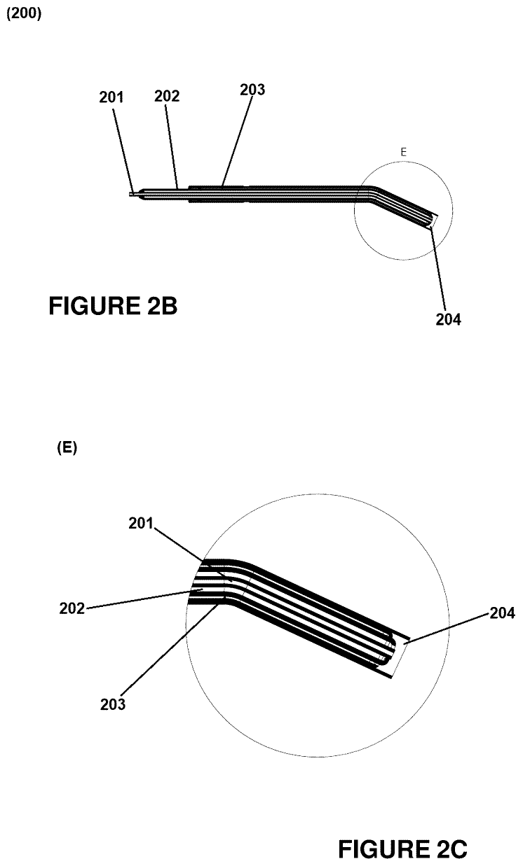

That is to say, whereas traditional nozzles comprise two concentric conduits, a central one for water and an outer one for air, the sprayer nozzle (200) of the present invention comprises a central conduit for water (201); an intermediate conduit for air (202); and an outer conduit for the passage of dental solutions (203), disposed concentrically and in continuation of the channels of the main body (100), as can be observed in detail in FIGS. 2B and 2C. Naturally, from a dimensional standpoint, the nozzle (200) of the present invention, shown in FIG. 2A, presents an enlarged diameter configuration as compared to conventional three-way syringe nozzles which typically are provided with only two pathways.

Likewise, with respect to the length of the tube (210) and the angle (220) of the nozzle (200), the same are maintained in conformity with the standard tips for conventional three-way syringes (300), which already set a standard for typical dental instrumentation. In this manner, the additional advantages of the present device are incorporated into a tool that is completely familiar to the dental practitioner. Nonetheless, owing to its interchangeable capability, other nozzles (200) with different tube lengths (210), angles (220), and geometries are perfectly adaptable. Thus, its replacement is carried out by removing the nozzle (200) to be set aside or sterilized and placing a new one in its place, mounting it to the main body (100) through the tip orifice (104), as seen in FIG. 1C. The main body (100) comprises a quick-fit mechanism, by means of which the nozzles (200) fit unequivocally in one position upon being introduced through the tip orifice (104). To this end, the nozzles comprise a groove (205) on the tube (210) that locks against corresponding protrusions disposed on the interior of the tip orifice (104). To remove the nozzles (200) for their replacement, it is sufficient to exert enough force to overcome the resistance of the protrusions against the groove (205) and simply withdraw them by pulling. This configuration allows the conduits of the main body to align perfectly with the orifices of the nozzle. As observed in FIG. 2A, the nozzle comprises an orifice (211), which is the inlet for the water channel; an orifice (212), which is the inlet for the compressed air channel; and an orifice (213), which is the inlet for the dental solution channel.

For this particular device, the diameter of the central conduit (201), intended for the passage of water, maintains a dimension similar to that of traditional nozzles, as its main function is to preserve the original water flow of the three-way syringe. In turn, the intermediate conduit (202) for compressed air has a larger diameter, such that the air can completely surround the water flow, generating a uniform spray when combined with the liquid.

Finally, the third conduit (203), intended for the passage of the dental solution, has a diameter specifically calculated to facilitate its suction via the Venturi effect, generated by the compressed air flow. The concentric disposition of this third conduit (203) relative to the other two is fundamental, as it is located in such a way that the dental solution mixes with the air at the precise moment of exit (204), which maximizes atomization and reduces the risk of splashing.

The geometry of these channels and their convergence points permit an optimal particle size in the nebulization, ensuring uniform and complete coverage of the treated surface. In this manner, the application of dental solutions becomes easier and more effective than by means of traditional applicators, as the nebulized spray allows for better reach and coverage of areas that are difficult to access for common syringes or cotton pellets.

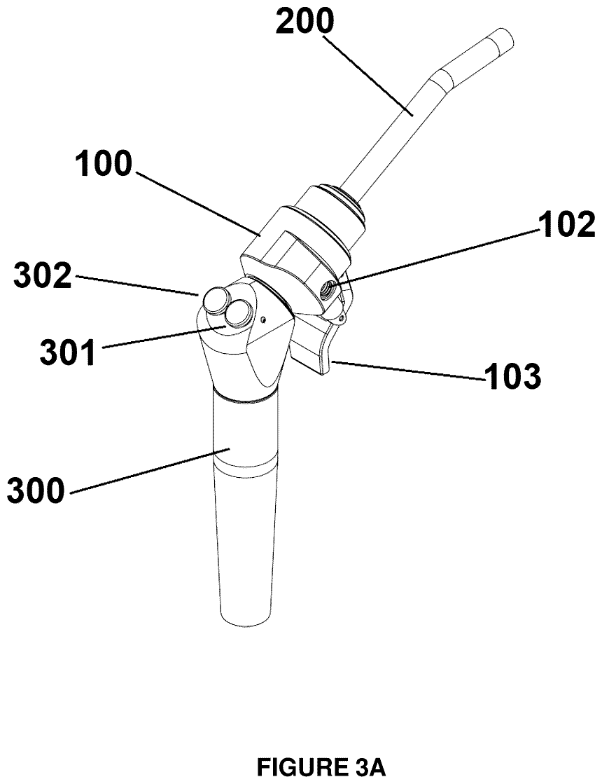

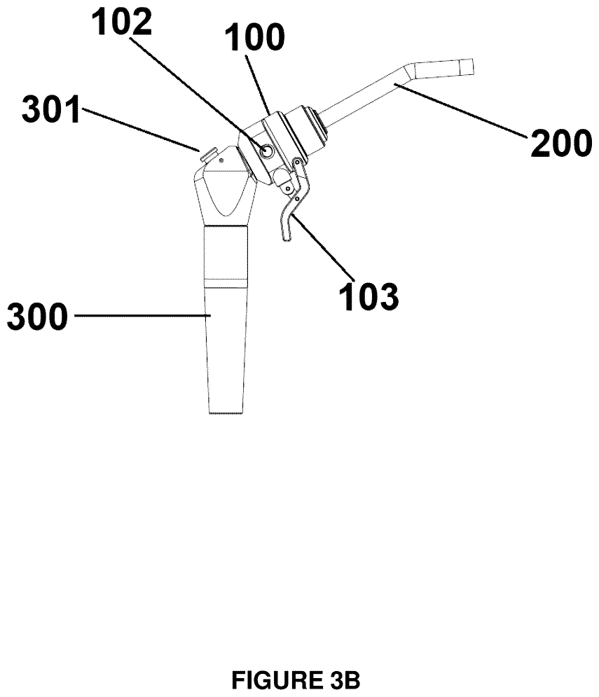

The dental sprayer device of the present invention incorporates, as shown in FIGS. 1A-C, on its main body (100), an ergonomic trigger (103) specifically designed to optimize control and precision in the administration of dental solutions, thereby providing the practitioner with modulated dosing adapted to the needs of each clinical procedure. This trigger (103) is strategically positioned on the device body (100), permitting the operator to manipulate it with the same hand that holds the three-way syringe (300), without the need to interrupt or change the hand position during treatment, as can be understood from FIGS. 3A-B. This ensures a continuous workflow in instrument handling, improving operative efficiency and reducing the time required for each procedure.

The ergonomic design of the trigger (103) has been developed to naturally accommodate the practitioner's index finger, enabling it to be actuated with minimal pressure while maintaining firm and precise control over the solution flow, in an intuitive and simple manner.

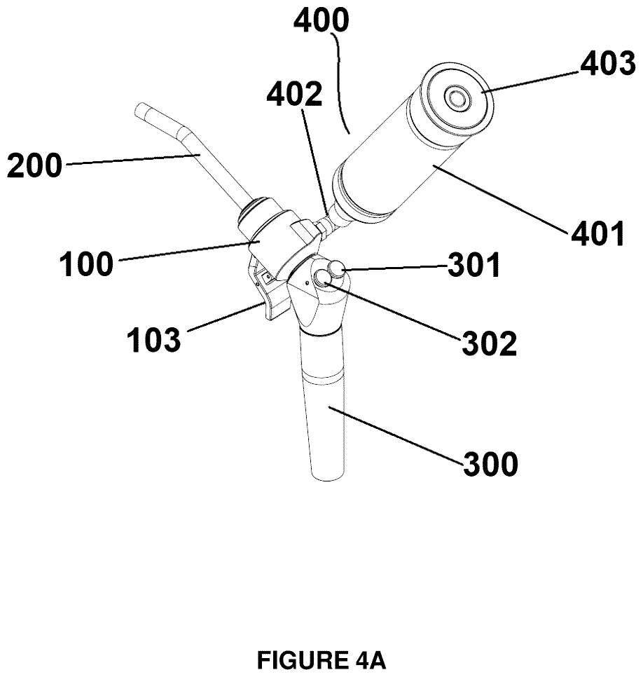

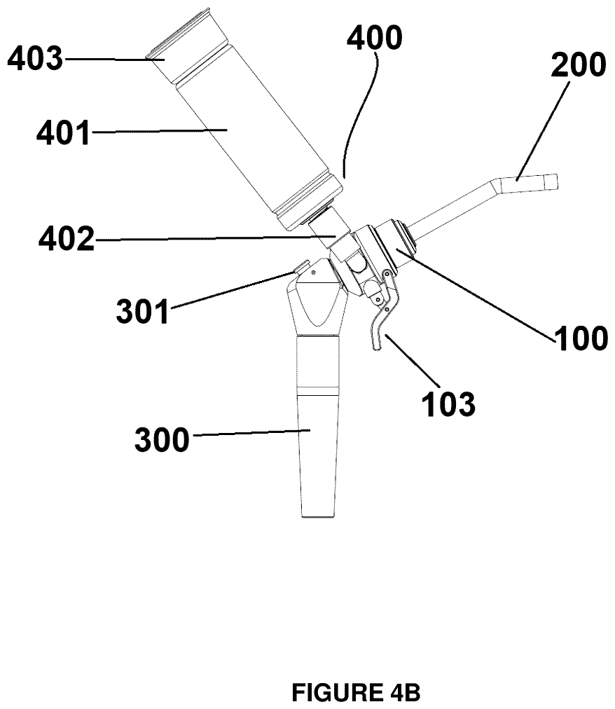

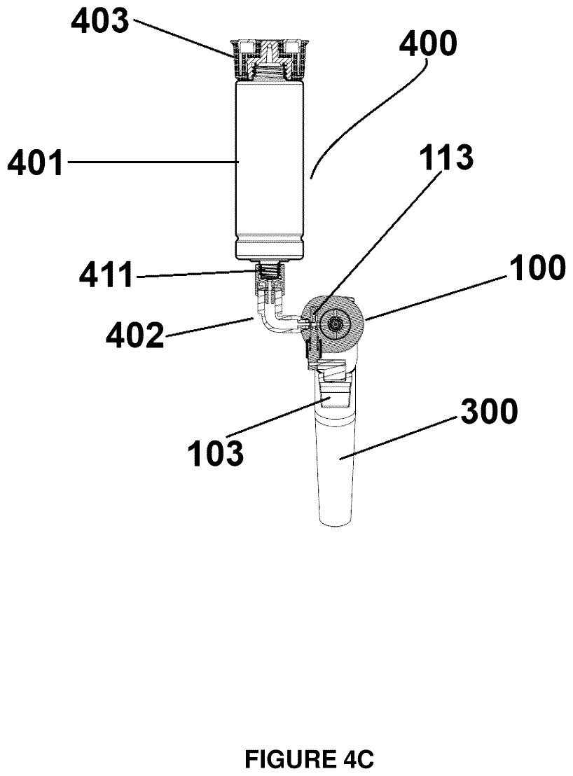

From a technical standpoint, the trigger (103) operates by means of a valve mechanism (113) integrated within the main body (100) of the device. This is depicted in detail in FIG. 4C.

This valve (113) is configured to administer the passage of the solution from the container (401) towards the administration conduit (203) in a precise and controlled manner. The valve (113) is a flow control valve, such that the pressure exerted on the trigger (103) determines the opening of said valve (113), thereby modulating the quantity of solution released and permitting the dental practitioner to adjust the flow in real-time, according to the quantity required for each application.

The dosing mechanism permits the egress of the solution to be constant and controlled, avoiding splashes or excesses that could irritate the surrounding tissues in the oral cavity. Furthermore, the system is designed such that the release of the solution stops immediately upon releasing the trigger (103), which not only prevents product waste but also eliminates the risk of dripping when the device is not in use.

This ergonomic trigger (103) design is particularly advantageous in the context of dental procedures that require a uniform and constant application of solutions over broad or delicate surfaces, such as the oral mucosa, where a homogeneous distribution is crucial for the effectiveness of the treatment. For example, during the application of desensitizing agents or anesthetic solutions, the practitioner can adjust the exact quantity of product, avoiding both excessive and insufficient application, which markedly improves the patient experience by reducing the likelihood of post-procedural discomfort or sensitivity.

This arrangement is especially practical as it allows the practitioner to comfortably manipulate the three-way syringe (300) without modifying their customary grip. While the traditional air (301) and water (302) controls of the three-way syringe remain operable by the thumb, allowing for the selective actuation of one, the other, or both simultaneously, the flow of the dental solution to be dispensed is controlled with the index finger, permitting all flow combinations, without restrictions.

For its part, the interchangeable container system (400) constitutes another practical and innovative aspect. It primarily comprises a container (401) integrated with the main body (100), by means of an adapter conduit (402) that couples to the inlet (102) disposed on said main body (100) for said purpose, as illustrated in detail in FIGS. 4A-C. This adapter conduit (402) is configured to receive specially designed containers (401) that contain the different dental solutions. Said containers (401) are provided with a threaded connection mechanism (411) or, alternatively, a quick-connect bayonet cam-type union that permits their installation and removal by means of a simple twist, while at the same time ensuring a hermetic seal during use. The containers (401) can be both disposable, pre-filled with specific products by manufacturers, and refillable, permitting the practitioner to use their own solutions. This results in an additional advantage, as it grants great versatility and considerably expands the possibilities of utilization and application, while at the same time reducing waste from the disposal of empty containers.

As previously mentioned, the dental sprayer device of the present invention utilizes a suction mechanism based on the Venturi effect to draw and atomize dental solutions from the container (401) towards the outlet (204) of the nozzle (200), permitting a nebulized and controlled application. In this manner, the need for additional pumping systems is eliminated, thereby simplifying the device design and improving its operability. To achieve the desired suction effect, the contents of the container (401) must be maintained at atmospheric pressure. For this reason, the containers (401) comprise, at their end opposite the threaded union (411) for coupling to the main body (100), a vent valve (403). This vent valve (403) allows the external and internal pressures of the container (401) to be equalized, preventing collapse due to the Venturi suction.

Once the container (401) is placed in the adapter conduit (402) and is in fluid communication with the interior of the main body (100) of the device, the vent valve (403) can be released simply by pulling it to place it in an open position. This configuration of the vent valve (403), which is initially presented closed and can be opened for its utilization, presents two clear advantages. Firstly, it serves as a method to ensure the tamper-evidence of the containers (401), when these are presented as disposable, pre-filled containers. Additionally, being able to present the vent valve (403) as initially closed prevents the premature oxidation of solutions sensitive to air contact. In this manner, the correct preservation of the substances contained in the containers is ensured for their transport or storage.

Naturally, by pressing the vent valve (403) towards the container (401) again, it can be closed, thereby preventing the free exchange of gases when the container (401) is not in use. That is to say, the vent valve (403) is operable to alternate between a closed position, wherein it prevents the passage of gases, and an open position, wherein it permits the free circulation of gases to and from the interior of the container (401).

In the sprayer device of the present invention, the compressed air flow, originating from the three-way syringe (300), is directed towards a narrowing in the internal conduit of the main body (100), thereby generating a low-pressure zone that suctions the dental solution from the container. This phenomenon permits the air flow, upon passing through this low-pressure zone, to draw the liquid from the container, transporting it towards the nozzle and combining it into an atomized and uniform flow.

The suction generated by means of the Venturi effect permits a nebulized dispersion of the solution, which is essential for procedures requiring uniform coverage of dental surfaces or mucosa. This finely atomized spray is especially useful for the application of desensitizing agents, topical anesthetics, and other products that necessitate an even and controlled distribution to ensure their effectiveness without causing irritation to the surrounding tissues.

An advantage of this mechanism is that it utilizes the compressed air already present in the three-way syringe (300), thereby achieving suction without additional moving parts or electric pumps, which reduces wear, maintenance costs, and the risk of failure in the device. This design permits the dental practitioner to maintain efficient and uninterrupted operation, by simply manipulating the air flow control (301) and the dosing trigger (103) to control the quantity of solution dispensed.

All components of the device that come into contact with the dental solutions are fabricated from inert and biocompatible materials, resistant to corrosion, and compatible with the disinfection and sterilization processes commonly used in dental practice. Preferably, surgical-grade stainless steel or anodized aluminum is used. The spray nozzle (200), in particular, is designed to be easily detachable, permitting its sterilization between patients in accordance with standard biosecurity protocols.

Furthermore, some components of the sprayer device, such as, for example, the ergonomic trigger (103) or the adapter conduit (402) for the containers (401), are customizable. In this way, it is possible to exchange them for parts with different finishes and/or colors, which grant an additional aesthetic appeal to the attachment, in addition to conferring a distinctive character that permits the visual identification of different tools that are in proximity to one another.

1. A sprayer device adaptable to a dental three-way syringe, comprising:
a main body (100) configured to couple to a conventional three-way syringe (300);
a nebulizing spray nozzle (200), which couples to said main body (100); and
a system of interchangeable containers (400) containing dental solutions;
wherein the main body (100) houses an arrangement of concentric channels,
wherein the central conduit is intended for the passage of water; the intermediate conduit is intended for the passage of compressed air; and the outer conduit is intended for the passage of dental solutions;
wherein said outer conduit for the passage of dental solutions is so arranged that the intermediate conduit for the passage of compressed air produces a Venturi suction effect;
wherein said main body (100) further comprises a hermetic coupling mechanism (101) that is configured to be coupled to a conventional three-way syringe (300) while maintaining the alignment of the compressed air and water conduits;
wherein the sprayer nozzle (200) comprises a central conduit for water (201); an intermediate conduit for air (202); and an outer conduit for the passage of dental solutions (203), disposed concentrically and in continuation of the channels of the main body (100); and wherein the system of interchangeable containers (400) comprises a container (401) integrated with the main body (100) by an adapter conduit (402) that couples to an inlet (105) disposed on said main body (100).

2. The sprayer device according to claim 1, wherein the main body (100) comprises an ergonomic trigger (103) which controls a valve (113) disposed within said main body (100) that permits or interrupts the dosing and flow of the dental solutions dispensed through the third conduit (203).

3. The sprayer device according to claim 2, wherein the valve (113) is a flow control valve, such that the pressure exerted on the trigger (103) determines the opening of said valve (113).

4. The sprayer device according to claim 1, wherein the main body (100) is fabricated from biocompatible materials resistant to sterilization processes, such as surgical-grade stainless steel, or anodized aluminum.

5. The sprayer device according to claim 1, wherein the nebulizing spray nozzle (200) is detachable and is fabricated from biocompatible materials resistant to sterilization processes, such as surgical-grade stainless steel, or anodized aluminum.

6. The sprayer device according to claim 1, wherein the system of interchangeable containers (400) includes a container (401) with a connection mechanism (411) for coupling to the adapter conduit (402), and a vent valve (403) to equalize the external and internal pressures of said container (401).

7. The sprayer device according to claim 6, wherein the connection mechanism (411) of the container (401) comprises a threaded union.

8. The sprayer device according to claim 6, wherein the connection mechanism (411) of the container (401) comprises a quick-connect bayonet cam-type union.

9. The sprayer device according to claim 1, wherein the vent valve (403) is operable to alternate between a closed position, wherein it prevents the passage of gases, and an open position, wherein it permits the free circulation of gases to and from the interior of the container (401).

10. The sprayer device according to claim 1, wherein the coupling mechanism (101) of the main body (100) to the three-way syringe (300) comprises a threaded union with O-ring type seals made of elastomeric material that maintain tightness at the connection points.

11. The sprayer device according to claim 1, wherein the coupling mechanism (101) of the main body (100) to the three-way syringe (300) comprises a press-fit union with O-ring type seals made of elastomeric material that maintain tightness at the connection points.

12. The sprayer device according to claim 1, wherein the nozzles (200) comprise a groove (205) on the tube (210) that lock against corresponding protrusions disposed on the interior of the tip orifice (104).
